(19) United States
(12) Patent Application Publication
(10) Pub. No.: US 2013/0144123 A1
(43) Pub. Date: Jun. 6, 2013

(54) PROPULSION ASSEMBLY FOR ENDOSCOPE AND FASTENING METHOD

(71) Applicant: FUJIFILM Corporation, Tokyo (JP)

(72) Inventors: Takayuki NAKAMURA, Ashigarakami-gun (JP); Naoyuki MORITA, Ashigarakami-gun (JP); Tsuyoshi ASHIDA, Ashigarakami-gun (JP); Nobuyuki TORISAWA, Ashigarakami-gun (JP); Takumi DEJIMA, Ashigarakami-gun (JP)

(73) Assignee: FUJIFILM CORPORATION, Tokyo (JP)

(21) Appl. No.: 13/688,304

(22) Filed: Nov. 29, 2012

(30) Foreign Application Priority Data
Dec. 1, 2011 (JP) 2011-263469

(51) Int. Cl. A61B 1/00

(52) U.S. Cl. CPC A61B 1/00133
USPC 600/114

(57) ABSTRACT

A propulsion assembly for an endoscope includes a support sleeve and an endless track device. A clamping sleeve is contained in the support sleeve. Male and female threads cooperate for moving the clamping sleeve between operative and non-operative states upon rotation of the clamping sleeve. The operative state is on a side of a proximal direction from the non-operative state along a central axis. A sealing device is contained in the support sleeve, has a C-shape in a resiliently deformable manner, is disposed around the endoscope and between the clamping sleeve and a receiving surface. The sealing device is pushed by the clamping sleeve when the clamping sleeve is in the operative state, for squeezing the endoscope by radially compressing thereabout, and released from push of the clamping sleeve when the clamping sleeve is in the non-operative state, for releasing the endoscope by radially returning to expand thereabout.

### BACKGROUND OF THE INVENTION

1. Field of the Invention

The present invention relates to a propulsion assembly for an endoscope and a fastening method. More particularly, the present invention relates to a propulsion assembly for an endoscope, which can be fastened to the endoscope reliably even by simple operation, and a fastening method for the propulsion assembly.

2. Description Related to the Prior Art

U.S.P. Ser. No. 2005/272,976 (corresponding to JP-A 2005-253892) discloses an endoscope used widely in the medical field. An elongated tube of the endoscope is entered in a body cavity of a patient, for example, gastrointestinal tract inclusive of large and small intestines. A wall of the gastrointestinal tract is imaged by the endoscope for diagnosis. Also, the endoscope is used for treatment of a lesion in the body cavity. The manipulation of the endoscope is very difficult typically for the gastrointestinal tract of a tortuous shape, for example, sigmoid colon which moves frequently to a large extent. The advance of the elongated tube in the sigmoid colon to a deep site requires high skill in the medical diagnosis for a doctor or operator. Simplification in the manipulation of the elongated tube even for the gastrointestinal tract of the tortuous shape is a highly important problem in the field of medical instruments.

A propulsion assembly for the endoscope has been developed recently. The propulsion assembly is fastened to a tip device of the elongated tube, and moves for advance of the tip device in the gastrointestinal tract. U.S. Pat. Nos. 6,971,990 and 7,736,300 (corresponding to JP-A 2009-513250) discloses the propulsion assembly, which includes a support sleeve mounted on the elongated tube, and an endless track device supported on the support sleeve for endlessly moving. The endless track device contacts a wall of the gastrointestinal tract and moves endlessly to propel the tip device of the elongated tube through the gastrointestinal tract by use of friction of the endless track device with the wall.

It is necessary to fasten the propulsion assembly firmly to the tip device without risk of drop from the endoscope in the course of the imaging. The endoscope can be used without the use of the propulsion assembly. It is preferable to construct the propulsion assembly in an externally settable manner with a removable property. Fastening of the propulsion assembly to the endoscope should be easy for quick operation. To this end, various mechanisms for fastening to meet the condition in the medical use has been suggested, for example, bayonet mechanism, engaging claws and the like. However, such mechanisms require a considerably large space for assembly. There occurs a problem of enlarging the size of the propulsion assembly which should be constructed in a reduced size in combination with the endoscope. Also, the claws should be formed together with the tip device for fastening of the propulsion assembly. Types of the endoscope for use with the propulsion assembly are limited. Conventional types of the endoscope cannot be used in combination with the propulsion assembly.

There is a suggestion of a method of fastening the tip device with an O-ring disposed in a through channel in the propulsion assembly for entry of the tip device. An screw of an annular shape is used and entered through a distal end of the through channel, to compress the O-ring set around the tip device, of which an inner surface is pressed against the tip device for fastening by the radial compression. There is a problem of smallness in the change of the diameter of the O-ring in the compression due to the property of the O-ring. A value range of the diameter of the tip device to be fastened reliably is not very large, so that the endoscope for which the fastening method is effective is limited. Also, it is necessary to enlarge an amount of the compression by use of the O-ring of a large thickness for higher strength of the fastening with a larger area of the contact. This is inconsistent to reduction of the diameter of the propulsion assembly and reduction of its length in the axial direction. Furthermore, considerably high force is required for compressing the O-ring. Operability of those elements for the purpose of fastening is rather low.

### SUMMARY OF THE INVENTION

In view of the foregoing problems, an object of the present invention is to provide a propulsion assembly for an endoscope, which can be fastened to the endoscope reliably even by simple operation, and a fastening method for the propulsion assembly.

In order to achieve the above and other objects and advantages of this invention, a propulsion assembly is provided, the propulsion assembly having an endless track device for endlessly moving in contact with a wall of a body cavity, for propulsion of a tip device of an endoscope in the body cavity. There is a support sleeve for mounting on the tip device, the endless track device extending around the support sleeve. A clamping sleeve is contained in the support sleeve. A male thread is formed on an outer surface of the clamping sleeve. A female thread is formed on an inner surface of the support sleeve, engaged helically with the male thread, for shifting the clamping sleeve between operative and non-operative states upon rotation of the clamping sleeve. A receiving surface is formed inside the support sleeve, and disposed on a proximal side from the clamping sleeve in the operative state. A sealing device is contained in the support sleeve, has a C-shape defined arcuately according to an annular shape in a resiliently deformable manner, is disposed around the tip device and between the clamping sleeve and the receiving surface, wherein the sealing device is pushed by the clamping sleeve when the clamping sleeve is in the operative state, for squeezing the tip device by radially compressing around the tip device, and released from push of the clamping sleeve when the clamping sleeve is in the non-operative state, for releasing the tip device by radially returning to expand around the tip device.

Furthermore, a first tapered surface is formed on an outer side of the sealing sleeve, and tapered relative to a central axis of the tip device. A second tapered surface is formed on an inner side of the clamping sleeve, tapered relative to the central axis, for pushing the first tapered surface when in the operative state, to compress the sealing device, and for coming away from the first tapered surface when in the non-operative state, to allow the sealing device to expand.

Furthermore, a mounting structure is disposed at an end of the clamping sleeve in a distal direction, for engagement with a jig device for rotating the clamping sleeve, to be supplied with torque.

In another preferred embodiment, furthermore, a first tapered surface is formed on an outer side of the sealing sleeve, and tapered relative to the central axis, wherein the first tapered surface, when the clamping sleeve is in the operative state, is pushed by the clamping sleeve for compressing the sealing device, and when the clamping sleeve is in the non-operative state, is released from the clamping sleeve coming away, to allow the sealing device to expand.

In still another preferred embodiment, furthermore, a tapered surface is formed on an inner side of the clamping sleeve, tapered relative to the central axis, for pushing the sealing device when in the operative state, to compress the sealing device, and for coming away from the sealing device when in the non-operative state, to allow the sealing device to expand.

Also, a fastening method for a propulsion assembly is provided, the propulsion assembly including an endless track device for endlessly moving in contact with a wall of a body cavity, for propulsion of a tip device of an endoscope in the body cavity, and a support sleeve for mounting on the tip device and for supporting the endless track device movably. The fastening method includes a step of entering the tip device in the support sleeve by entry of the tip device in a sealing device contained in the support sleeve and having a C-shape in a manner deformable resiliently in a radial direction. The clamping sleeve is rotated in the support sleeve by use of helical engagement between a female thread formed on an inner surface of the support sleeve and a male thread formed on an outer surface of the clamping sleeve, to move the clamping sleeve in a proximal direction along a central axis of the tip device. The sealing device is pushed with the clamping sleeve moved in the proximal direction, to compress the sealing device in the radial direction around the tip device, to squeeze the tip device.

The sealing device includes a first tapered surface at an end thereof in a distal direction, and the clamping sleeve includes a second tapered surface at an end thereof in the proximal direction for pushing the first tapered surface.

A jig device is used and accesses a distal portion of the propulsion assembly, to rotate the clamping sleeve relative to the support sleeve.

The clamping sleeve has a mounting structure disposed at an end thereof in a distal direction, and the jig device includes a rotatable driving head, engaged with the mounting structure, for applying torque thereto.

The driving head includes a plurality of first teeth, and the mounting structure includes a plurality of second teeth, meshed with the first teeth, for transmitting the torque.

Also, a fastening system for an endoscope is provided, and includes a propulsion assembly and a jig device. The propulsion assembly includes an endless track device for endlessly moving in contact with a wall of a body cavity, for propulsion of a tip device of the endoscope in the body cavity. There is a support sleeve for mounting on the tip device and for supporting the endless track device movably. A clamping sleeve is contained in the support sleeve. A male thread is formed on an outer surface of the clamping sleeve. A female thread is formed on an inner surface of the support sleeve, engaged helically with the male thread, for moving the clamping sleeve between operative and non-operative states upon rotation of the clamping sleeve, the operative state being on a side of a proximal direction from the non-operative state along a central axis of the tip device. A receiving surface is formed inside the support sleeve, and disposed on a side of the proximal direction from the clamping sleeve. A sealing device is contained in the support sleeve, has a C-shape defined arcuately according to an annular shape in a resiliently deformable manner, is disposed around the tip device and between the clamping sleeve and the receiving surface, wherein the sealing device is pushed by the clamping sleeve when the clamping sleeve is in the operative state, for squeezing the tip device by radially compressing around the tip device, and released from push of the clamping sleeve when the clamping sleeve is in the non-operative state, for releasing the tip device by radially returning to expand around the tip device. In combination, the jig device accesses a distal portion of the propulsion assembly, to rotate the clamping sleeve relative to the support sleeve.

Consequently, the propulsion assembly can be fastened to the endoscope reliably even by simple operation, because the sealing device for fastening can be compressed easily by use of the clamping sleeve.

### BRIEF DESCRIPTION OF THE DRAWINGS

The above objects and advantages of the present invention will become more apparent from the following detailed description when read in connection with the accompanying drawings, in which:

FIG. 1 is an explanatory view illustrating an endoscope and a propulsion assembly in combination;

FIG. 2 is a perspective view illustrating the propulsion assembly of which an endless track device is developed;

FIG. 3 is an exploded perspective view illustrating the propulsion assembly;

FIG. 4 is an exploded perspective view illustrating a drive sleeve, torque wire devices and motors;

FIG. 5 is a vertical section illustrating the propulsion assembly;

FIG. 6 is an explanatory view in a cross section illustrating the endless track device;

FIG. 7 is a perspective view illustrating a sealing device and a clamping sleeve for fastening to the endoscope;

FIG. 8 is a perspective view illustrating a screw driving device for rotating the clamping sleeve.

### DETAILED DESCRIPTION OF THE PREFERRED EMBODIMENT(S) OF THE PRESENT INVENTION

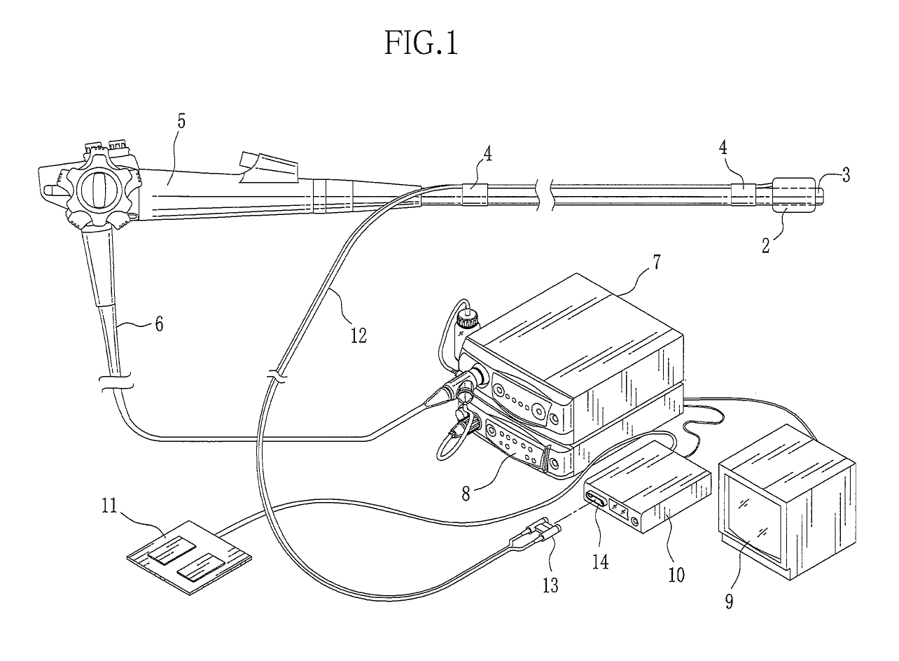

In FIG. 1, a propulsion assembly 2 is for use with an endoscope. The propulsion assembly 2 is fitted around a tip device 3 of the endoscope. The endoscope includes an image sensor, lighting windows, a steering device, an elongated tube, a handle 5, steering wheels and the like. The image sensor is incorporated in the tip device 3, and is a CCD or CMOS image sensor. The lighting windows are formed in the tip device 3 and emit light. The image sensor images an object in a body cavity illuminated with the light from the lighting windows, such an object as a wall of a stomach or intestine of a gastrointestinal tract of a patient. The steering device is disposed at a proximal end of the tip device 3 for steering to enter the tip device 3 in the body cavity to reach the object. The propulsion assembly 2 operates to facilitate the entry of the tip device 3. The steering wheels are disposed on the handle 5, and manually rotated to operate the steering device for bending up and down and to the right and left.

The handle 5 includes a button and an end sleeve . The button is operable to change over the supply and suction of air or water. The end sleeve has an instrument opening where a biopsy forceps or other medical device is advanced. A universal cable 6 extends from the handle 5, and connected to alight source apparatus 7 and a processing apparatus 8. Light from a lamp in the light source apparatus 7 is guided by a light guide fiber extending through the universal cable 6 and the endoscope to the lighting windows.

The processing apparatus 8 processes an image signal from the universal cable 6 in the signal processing suitably. A display panel 9 is driven to display the image of the image signal. The processing apparatus 8 discerns the type information of the endoscope for use according to the input information from the endoscope through the universal cable 6. The processing apparatus 8 automatically changes over the control and/or display suitably according to the type information, typically if the control with differences for the types is required in the course of the manipulation, or if the display with differences for the types is required on the display panel 9.

An actuating apparatus 10 or controller is connected with the processing apparatus 8 electrically. The actuating apparatus 10 actuates and controls the propulsion assembly 2. A wire sheath 12 of a dual lumen form extends from a proximal end of the propulsion assembly 2. An adhesive tape 4 or surgical tape positions the wire sheath 12 on the elongated tube of the endoscope at suitable points. The wire sheath 12 extends properly into the body cavity even upon moving the endoscope into the body cavity or during the manipulation.

First and second torque wire devices are disposed to extend discretely through the wire sheath 12. Distal end portions of the wire devices are coupled to a driving mechanism (sleeve) of the propulsion assembly 2. The wire devices are flexible but have high torsional rigidity so that torque applied to their proximal end are transmitted by those to their distal end substantially without attenuation. A key coupling device 13 for plug-in is disposed at the proximal end of the wire devices. A rotating coupling 14 for plug-in is disposed in the actuating apparatus 10, and connected mechanically with the key coupling device 13. First and second motors are incorporated in the actuating apparatus 10. When the key coupling device 13 is plugged to the rotating coupling 14, each of the wire devices is ready to rotate with one of the first and second motors.

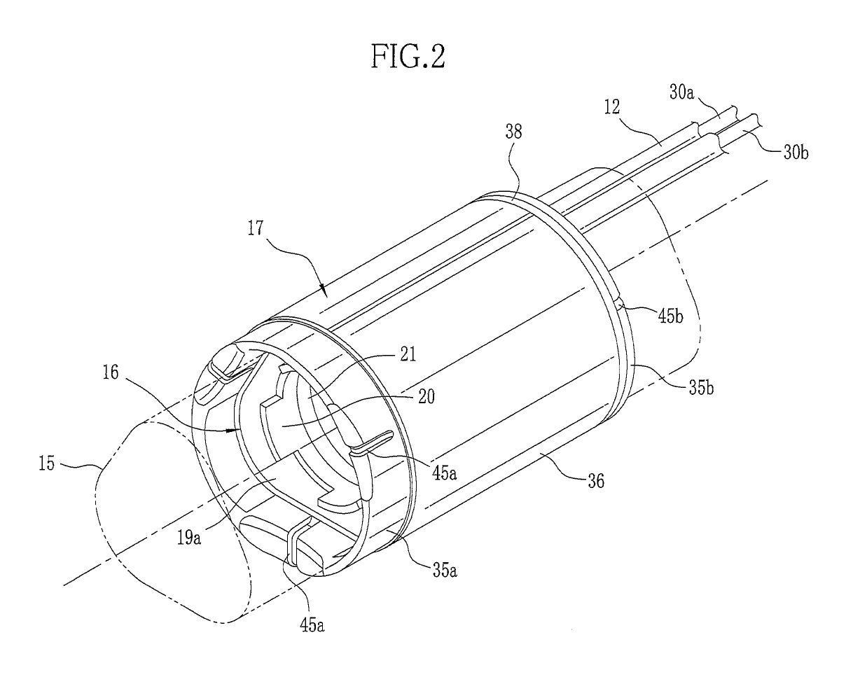

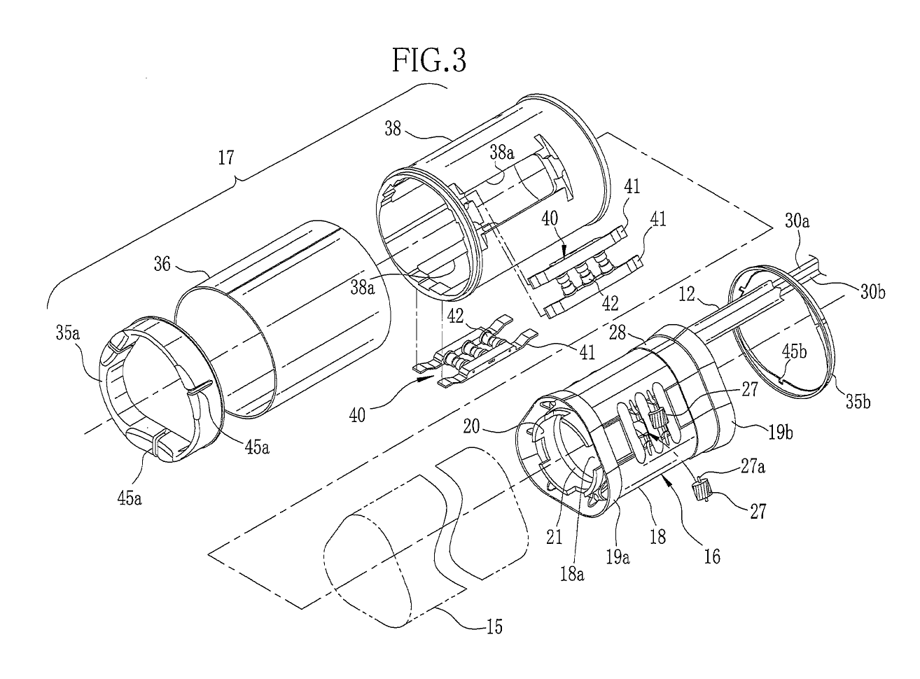

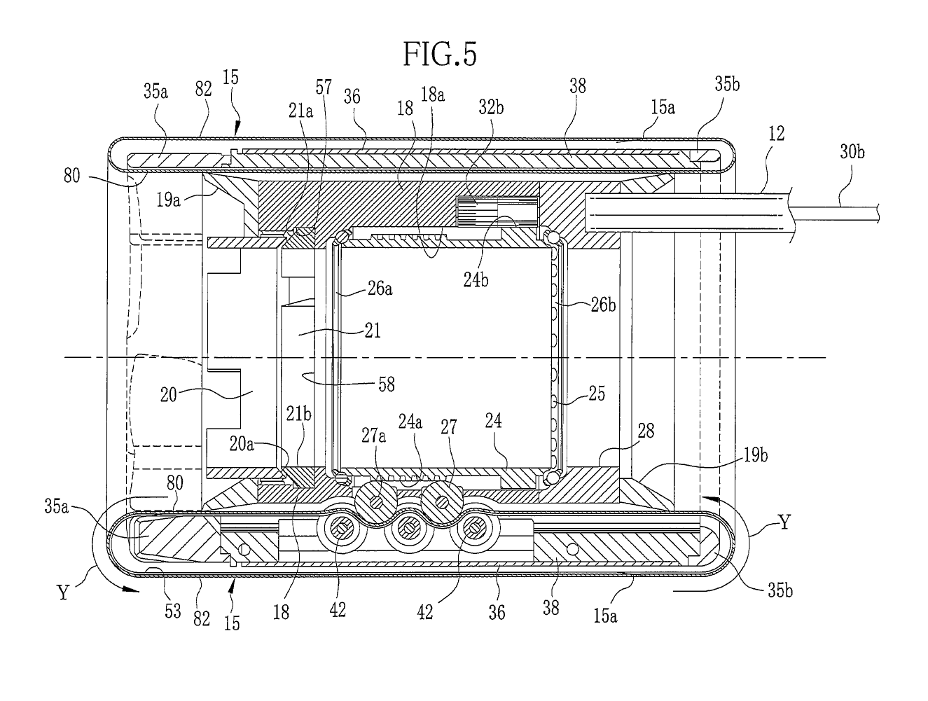

The propulsion assembly 2 is used effectively specially for colonoscopy, because of manipulation for advance and pull in the sigmoid colon or transverse colon. The propulsion assembly 2 is substantially cylindrical. An endless track device 15 or membrane or toroidal device is disposed on the outside of the propulsion assembly 2, is constituted by a flexible sheet of synthetic resin with sufficient rigidity. In FIGS. 2 and 3, the endless track device 15 is depicted in a developed form of a sleeve for understanding. A final form of the endless track device 15 is in a ring shape or toroidal shape after connecting front and rear ends of the sleeve. The endless track device 15 has an annular surface. See FIG. 5. In FIGS. 2-5 and 7, a distal side for protruding the tip device 3 is depicted on the left side. A proximal side near to the handle 5 of the endoscope is depicted on the right side.

In FIGS. 2 and 3, the propulsion assembly 2 includes an inner sleeve unit 16 and an outer sleeve unit 17. The inner sleeve unit 16 is disposed inside the endless track device 15. The outer sleeve unit 17 is disposed around the inner sleeve unit 16. The inner sleeve unit 16 includes a support sleeve 18, a cap ring 28, a distal cover flange 19a for wiping, a proximal cover flange 19b for wiping, a clamping sleeve 20 or collet sleeve, a sealing device 21 (in a C-shape) or C-ring or collet head, and a drive sleeve 24. The support sleeve 18 has a cylindrical inner surface and an outer surface in a shape of a triangular prism. A through channel 18a is defined in the support sleeve 18. The cap ring 28 is in a triangular shape, and retained to a proximal end of the support sleeve 18 by a screw, press-fit or caulking. The cover flanges 19a and 19b are attached to respectively the distal end of the support sleeve 18 and the proximal end of the cap ring 28. The clamping sleeve 20 is helically engaged with a thread formed inside the support sleeve 18, and rotates to move in the axial direction. The sealing device 21 is formed from synthetic resin with resiliency, and has a diameter changeable by movement of the clamping sleeve 20 in the axial direction. The drive sleeve 24 is a driving mechanism supported inside the support sleeve 18 in a rotatable manner.

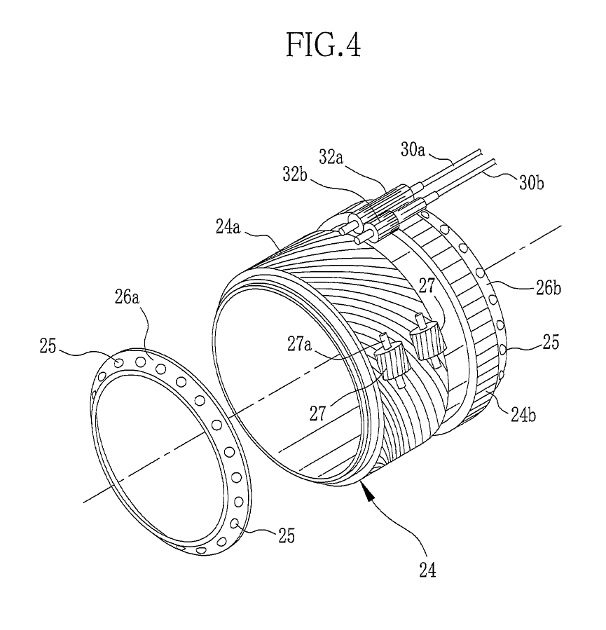

In FIG. 4, the propulsion assembly 2 includes bearing rings 26a and 26b, each of which is constituted by plural bearing balls 25 arranged annularly. The bearing rings 26a and 26b support ends of the drive sleeve 24 on an inner surface of the support sleeve 18 in a rotatable manner. The cap ring 28 is secured to a proximal end of the support sleeve 18, and prevents the drive sleeve 24 from dropping out. Worm gear teeth 24a or thread, and spur gear teeth 24b are arranged on an outer surface of the drive sleeve 24. Two rotatable worm wheels 27 or helical gears are supported on the support sleeve 18, and meshed with the worm gear teeth 24a through openings in the support sleeve 18. Three pairs of the worm wheels 27 are arranged equiangularly from one another around the drive sleeve 24. When the drive sleeve 24 rotates, the worm wheels 27 rotate around a gear shaft 27a in the same direction simultaneously.

A distal end of the wire sheath 12 is attached to the inside of the proximal end of the cap ring 28 by use of adhesion or thermal welding. Distal ends of first and second torque wire devices 30a and 30b protruding from the wire sheath 12 extend to pass through holes in the cap ring 28. First and second coupling gears 32a and 32b or pinions are firmly connected with distal ends of the wire devices 30a and 30b. As illustrated in the drawing, rotational shafts protrude from respectively the coupling gears 32a and 32b as rotational centers. The shafts are received in holes formed in the support sleeve 18, to keep the coupling gears 32a and 32b rotatable.

Only the first coupling gear 32a of the first wire device 30a is meshed with the spur gear teeth 24b of the drive sleeve 24. The second coupling gear 32b of the second wire device 30b is meshed with the first coupling gear 32a but not with the spur gear teeth 24b. Thus, the drive sleeve 24 is driven by rotation of the first coupling gear 32a in connection with the first wire device 30a. However, the wire devices 30a and 30b are driven by torques generated by respectively the motors. The second coupling gear 32b is rotated in a direction opposite to that of the first coupling gear 32a. The torque from the second wire device 30b is added to the torque of the first coupling gear 32a, so that the drive sleeve 24 can be rotated with a high torque.

Each of the cover flanges 19a and 19b includes a flange edge shaped to increase a width in the axial direction. The flange edge receives an inner surface of the endless track device 15 with closeness while the endless track device 15 turns around. The flange edge prevents various materials from pull into the propulsion assembly 2 together with the moving outer surface of the endless track device 15, the materials including foreign material and tissue of a body part.

The propulsion assembly 2 is firmly fastened to the tip device 3 by receiving entry in the through channel 18a of the support sleeve 18. Initially, the tip device 3 is entered in the proximal end of the propulsion assembly 2. A distal end of the tip device 3 is positioned to protrude from a distal end of the propulsion assembly 2 before fastening the propulsion assembly 2. When the clamping sleeve 20 is moved in the axial direction by rotation for helical engagement, a second tapered surface 20a of the clamping sleeve 20 in FIG. 5 pushes a first tapered surface 21a of the sealing device 21, which is compressed to decrease its diameter. Accordingly, an inner surface of the sealing device 21 is tightly pressed against an outer surface of the tip device 3 to squeeze the tip device 3 in the through channel 18a in the support sleeve 18.

The outer sleeve unit 17 includes a distal support ring 35a or bumper ring, a cover sheet 36 for shielding, a guide sleeve 38 for supporting rollers, and a proximal support ring 35b or bumper ring, in a sequence in the proximal direction. The outer sleeve unit 17 is combined with the inner sleeve unit 16 and the endless track device 15 according to the steps as follows.

In FIGS. 2 and 3, a sheet material for the endless track device 15 in a developed form is formed in a cylindrical shape. The inner sleeve unit 16 is positioned so that its outer surface is covered inside the cylindrical shape of the sheet material. The inner sleeve unit 16 with the endless track device 15 is entered in the guide sleeve 38. Three holder openings 38a are formed in the guide sleeve 38 to extend in the axial direction, and arranged equiangularly from one another with 120 degrees. Roller mechanisms 40 are mounted in respectively the holder openings 38a.

The roller mechanisms 40 include three idler rollers 42, and a pair of roller supports 41 or frames for supporting the idler rollers 42 in alignment. The roller supports 41 are resilient thin plates of metal, and are fixed to the guide sleeve 38 by fitting their ends in end portions of the holder openings 38a. A center of the roller supports 41 in the longitudinal direction becomes curved to enter an inner space in the guide sleeve 38 through the holder openings 38a. The idler rollers 42 supported by the roller supports 41 press the endless track device 15 toward the worm wheels 27 owing to the curved form of the roller supports 41. As a result, the endless track device 15 is tensioned tightly between the worm wheels 27 and the idler rollers 42. See FIG. 5. There is degree of freedom in one of the idler rollers 42 disposed at the center in relation to the longitudinal direction of the roller supports 41, because the center roller is supported by the opening extending longitudinally. A relative position of the endless track device 15 to two lateral rollers included in the idler rollers 42 is automatically adjusted for supporting the endless track device 15 with the worm wheels 27 in an optimally balanced manner.

The roller mechanisms 40 are fitted in the holder openings 38a fixedly on the guide sleeve 38. The idler rollers 42 project to the inside of the guide sleeve 38 and keep the guide sleeve 38 immovable in the axial direction relative to the inner sleeve unit 16. The endless track device 15 is tensioned while the roller mechanisms 40 are combined with the guide sleeve 38. The support rings 35a and 35b are fixed to respectively the distal and proximal ends of the guide sleeve 38. Three grooves 45a are formed in the distal support ring 35a. Three grooves 45b are formed in the proximal support ring 35b. The grooves 45a and 45b are aligned with the roller mechanisms 40 in the axial direction. The cover sheet 36 tightly covers the outer surface of the guide sleeve 38 together with the roller mechanisms 40.

The sleeve of the endless track device 15 in a developed form is positioned between the inner and outer sleeve units 16 and 17. Those units are combined with one another, before ends of the sleeve of the endless track device 15 are turned over and connected with one another. A joint portion 15a of the endless track device 15 is formed. Note that inclinations can be preferably formed with ends of the sleeve of the endless track device 15, so that the joint portion 15a can have a small thickness without an excessive unevenness of the thickness. In FIG. 5, an assembled structure of the propulsion assembly 2 is schematically illustrated. The endless track device 15 can have an inner space to wrap the outer sleeve unit 17 entirely in the toroidal shape. It is possible to fill the inner space with suitable fluid, such as air, physiological saline water, colloid of synthetic resin, oil, grease, lubricant fluid of various types, and the like.

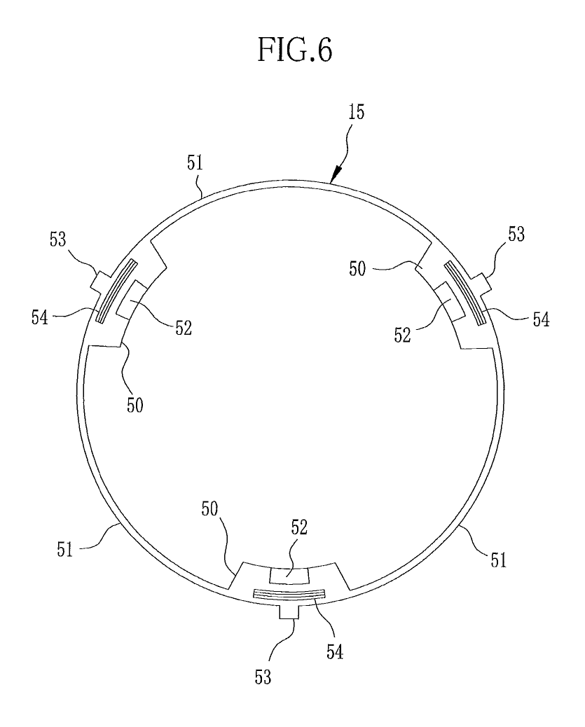

In FIG. 6, a sleeve for forming the endless track device 15 is viewed in a cross section. The endless track device 15 is constituted by a multi-layer sheet of polyurethane resin or the like with plural film layers. Three reinforcing ridges 50 are formed on an inner sleeve surface of the endless track device 15, arranged equiangularly from one another, and formed in a trapezoidal shape as viewed in section. The reinforcing ridges 50 have a larger thickness than a membrane wall 51, and are constituted by a sheet of plural film layers of a higher number than those in the membrane wall 51. The reinforcing ridges 50 extend longitudinally in the axial direction. Engaging teeth 52 or rack gear teeth are disposed on the surface of the reinforcing ridges 50, and arranged with an inclination for mesh with the worm wheels 27. Alignment ridges 53 are formed on the endless track device 15, extend longitudinally, and are opposite to the reinforcing ridges 50. Also, a mesh sheet 54 of fiber is disposed between the engaging teeth 52 and each of the alignment ridges 53.

The endless track device 15 is used in the toroidal shape in FIG. 5. The three reinforcing ridges 50 are nipped between the worm wheels 27 and the idler rollers 42. The worm wheels 27 are meshed with the engaging teeth 52. Rotation of the worm wheels 27 is transmitted directly to the endless track device 15 by the engaging teeth 52. The endless track device 15 can turn around efficiently in the axial direction. The reinforcing ridges 50 and also the mesh sheet 54 are in the multi-layer form. The engaging teeth 52 in the endless track device 15 can have sufficient mechanical strength even upon receiving driving force directly from the worm wheels 27, because the engaging teeth 52 do not deform or the endless track device 15 does not break. Also, the membrane wall 51 disposed beside the reinforcing ridges 50 is effective in reducing resistance of the endless track device 15 during passage between the inner and outer sleeve units 16 and 17.

Roller grooves are formed in respectively the idler rollers 42 at the center. The alignment ridges 53 disposed opposite to the reinforcing ridges 50 are engaged with the roller grooves when the endless track device 15 moves. Note that the outer sleeve unit 17 can be constructed in an adjustable form for reducing the inner space of the endless track device 15 in a tightly wrapped condition. In this form, the alignment ridges 53 are engaged also with the grooves 45a and 45b of the support rings 35a and 35b. The alignment ridges 53 are effective in stabilizing the path of the movement, as the endless track device 15 can be prevented from shifting in a zigzag manner while moved in the axial direction.

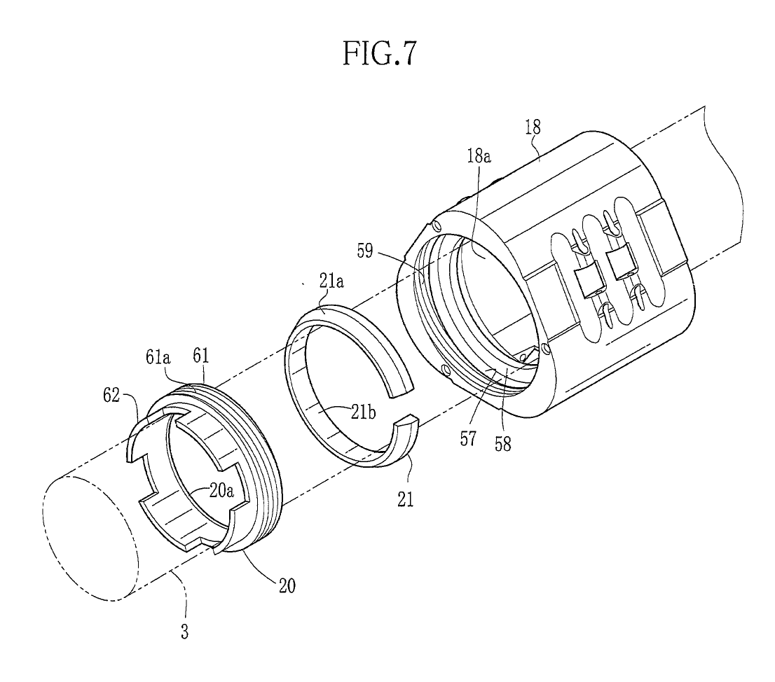

In FIG. 7, the clamping sleeve 20 and the sealing device 21 are arranged in the through channel 18a of the support sleeve 18. Note that elements of the propulsion assembly 2 other than the support sleeve 18, the clamping sleeve 20 and the sealing device 21 are omitted from FIG. 7.

The sealing device 21 is in the C-shape as a portion of a ring, and formed from synthetic resin or other resilient material. The sealing device 21 has an outer diameter slightly larger than an inner diameter of the through channel 18a, and is shaped in a trapezoid form as viewed in a cross section. The first tapered surface 21a of the sealing device 21 has a diameter increasing in the proximal direction. The sealing device 21 is disposed in the through channel 18a, and receives entry of the tip device 3.

The sealing device 21 keeps the propulsion assembly 2 positioned on the tip device 3. An inner surface 21b of the sealing device 21 is pressed against the tip device 3 by the clamping sleeve 20 in a closed position for fastening. The sealing device 21 is deformable between an open position and the closed position. When the sealing device 21 is in the open position, the propulsion assembly 2 is released from the tip device 3 by removing the pressure for entry and removal of the tip device 3. The inner surface 21b is parallel to the axial direction of the tip device 3, and fully contacts the outer surface of the tip device 3 upon the compression of the sealing device 21.

The inner surface 21b of the sealing device 21 is kept in tight contact with the outer surface of the tip device 3, so that the propulsion assembly 2 is fastened to the tip device 3 with a considerably large area of the contact. This is effective in tightening the fastened state. Also, the feature of the sealing device 21 is advantageous in reducing the diameter of the propulsion assembly 2 and its length in the axial direction, because the sealing device 21 has the diameter slightly larger than the tip device 3 and a very small size in the axial direction.

An inner groove 57 is formed in a surface of the through channel 18a of the support sleeve 18, extends annularly, and is disposed on a distal side from the set position of the drive sleeve 24. The inner groove 57 is formed by enlarging the diameter of the through channel 18a. The sealing device 21 is resiliently deformed to decrease the diameter, and fitted in the inner groove 57 by entry in the through channel 18a. The sealing device 21 in the inner groove 57 is released to expand or increase the diameter by its property for recover. A depth of the inner groove 57 is smaller than a thickness of the sealing device 21 in a radial direction. At least one portion of the first tapered surface 21a protrudes from the inner groove 57 in the through channel 18a.

A receiving surface 58 is defined in the inner groove 57, is disposed at its proximal end, and contacts the sealing device 21 for positioning in the axial direction. The sealing device 21, while pushed by the clamping sleeve 20, is prevented by the receiving surface 58 from moving in the proximal direction. The sealing device 21 is squeezed between the clamping sleeve 20 and the receiving surface 58. The receiving surface 58 is annular and has a size to reduce an inner diameter of the through channel 18a toward the center. The sealing device 21 is prevented from moving in the proximal direction even when compressed in the radial direction.

The clamping sleeve 20 is a plastic part, and includes a threaded ring 61 or hollow screw, and a mounting structure 62 with a set of teeth disposed at the distal end of the threaded ring 61. A male thread 61a is formed around the threaded ring 61. A proximal end of the threaded ring 61 is in a trapezoid shape as viewed in a cross section, so that the second tapered surface 20a is formed to enlarge its inner diameter in the proximal direction in correspondence with the first tapered surface 21a of the sealing device 21. A female thread 59 is formed inside the through channel 18a and disposed on the distal side. The clamping sleeve 20 engages the male thread 61a with the female thread 59 helically, and is mounted movably in the through channel 18a.

The coupling between the threads 59 and 61a causes the clamping sleeve 20 to move in the proximal or distal direction through the through channel 18a according to one of the two rotational directions of the clamping sleeve 20. For fastening of the tip device 3, the clamping sleeve 20 is moved in the proximal direction to an operative state (operative mode position) . For moving the tip device 3 into or out of the through channel 18a, the clamping sleeve 20 is moved from the operative state in the distal direction to a non-operative state (non-operative mode position) .

The operative state is for securing the tip device 3 in the closed position (compression) of the sealing device 21. When the clamping sleeve 20 is moved in the proximal direction, the second tapered surface 20a pushes the first tapered surface 21a of the sealing device 21 for radial compression with a smaller diameter. The inner surface 21b of the sealing device 21 is pressed against the outer surface of the tip device 3. The sealing device 21 is squeezed between the clamping sleeve 20 and the receiving surface 58 to fasten the propulsion assembly 2 to the tip device 3. An amount of deforming the sealing device 21 sufficiently for fastening the tip device 3 is different according to the diameter of the tip device 3. Thus, the operative state of the clamping sleeve 20 for fastening is determined according to the diameter of the tip device 3. The sealing device 21 can be deformed by suitably adjusting a gap size of its C-shape, and sizes and inclinations of the tapered surfaces 20a and 21a. So the sealing device 21 can operate even for the diameter of the tip device 3 of various values. Note that only either one of the surfaces 20a and 21a can be tapered in the combination of the clamping sleeve 20 and the sealing device 21, for the purpose of converting the force in the axial direction into force in the radial direction.

The non-operative state is for release (open position) of the sealing device 21 from the tip device 3 by its radial expansion of return. When the clamping sleeve 20 is shifted from the operative state in a distal direction, the first tapered surface 21a of the sealing device 21 is released from push of the second tapered surface 20a. Thus, the sealing device 21 is expanded and returned radially to unfasten the tip device 3.

In the above embodiment, the sealing device 21 can be used repeatedly. However, the sealing device 21 may be a type for a single use. Although the sealing device 21 of the embodiment returns to the initial shape upon its release from the clamping sleeve 20, the sealing device 21 can have rigidity without a property for returning to its initial shape. The sealing device 21 can expand with such a small increase in the diameter for return as to allow withdrawal of the tip device 3 of the endoscope.

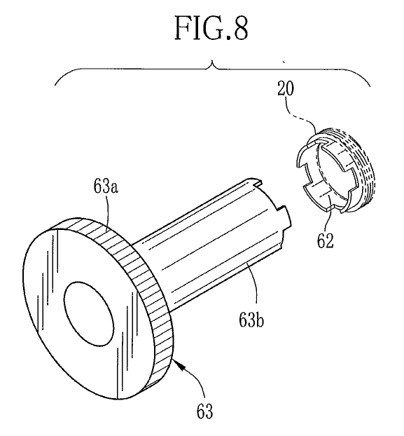

The mounting structure 62 has teeth (and recesses) arranged equiangularly from one another. In FIG. 8, a screw driving device 63 or jig device is used for entry in the proximal direction toward the mounting structure 62 for engagement. The screw driving device 63 is a single piece including a driving head 63b and a thumb wheel portion 63a. The driving head 63b has teeth (and recesses) in a pattern complementary to the mounting structure 62. When the thumb wheel portion 63a is rotated manually upon engagement of the driving head 63b with the mounting structure 62, the clamping sleeve 20 is rotated.

Note that all of inner diameters of the sealing device 21, the receiving surface 58 in the through channel 18a, the clamping sleeve 20 and the driving head 63b are larger than the diameter of the tip device 3, so that the tip device 3 can be entered smoothly.

The operation of the above construction is described now. The propulsion assembly 2 is fastened to the endoscope in such a manner that the tip device 3 partially protrudes in the distal direction. At first, the tip device 3 is entered in the through channel 18a through a proximal end of the propulsion assembly 2. The tip device 3 passes through the clamping sleeve 20 or collet sleeve, and protrudes from the distal end of the propulsion assembly 2 at a suitable size.

Then the distal end of the tip device 3 is entered in a central hole in the driving head 63b of the screw driving device 63 (jig device). The teeth of the driving head 63b are meshed with those of the mounting structure 62 of the clamping sleeve 20. Then the thumb wheel portion 63a of the screw driving device 63 is rotated manually with a hand, to rotate the clamping sleeve 20 in the clockwise direction.

The male thread 61a is helically engaged with the female thread 59 formed inside the through channel 18a at the distal end. Rotation of the clamping sleeve 20 in the clockwise direction shifts the clamping sleeve 20 internally in the proximal direction. The second tapered surface 20a presses the first tapered surface 21a of the sealing device 21 or C-ring or collet head. The sealing device 21 contacts the receiving surface 58 and does not shift in the proximal direction. The first tapered surface 21a of the sealing device 21 is tapered to increase the diameter in the proximal direction, and thus is pushed by the second tapered surface 20a to compress the sealing device 21 to decrease its diameter. The sealing device 21 is in the closed position to squeeze the tip device 3 in the through channel 18a. The sealing device 21 is pushed between the clamping sleeve 20 and the receiving surface 58. The tip device 3 is squeezed by the sealing device 21 inside the support sleeve 18. Thus, the propulsion assembly 2 is fastened to the tip device 3 reliably.

As the inner surface 21b of the sealing device 21 is entirely pressed against the outer surface of the tip device 3, the tip device 3 contacts the sealing device 21 with a large area. Thus, the propulsion assembly 2 is fastened to the tip device 3 firmly. To this end, the sealing device 21 is compressed to decrease the diameter against its rigidity. The tip device 3 can be fastened to the propulsion assembly 2 easily without large force of rotating the clamping sleeve 20 by manipulation.

After the screw driving device 63 is removed from the clamping sleeve 20, the wire sheath 12 extending from the proximal end of the propulsion assembly 2 is positioned along the outer surface of the steering device and the flexible device of the endoscope. Plural indicia are disposed on the wire sheath 12 equidistantly from one another, and indicate positions of attachment of the adhesive tape 4. The wire sheath 12 is attached to the steering device and the flexible device by use of the adhesive tape 4 according to the indicia. The key coupling device 13 at the proximal end of the wire sheath is plugged to the rotating coupling 14 for connection to the actuating apparatus 10, which is powered.

The actuating apparatus 10 checks whether the key coupling device 13 is plugged to the rotating coupling 14 or not upon powering. If it is judged that the plugging is improper or if the plugging is not detected, alarm information is emitted, for example, alarm sound or a visible alarm signal with light. If it is judged that the plugging is proper, a sensor in the rotating coupling 14 reads type information of the propulsion assembly 2 from a signal region disposed on a bridge portion of the key coupling device 13. According to the type information, the actuating apparatus 10 automatically determines a rotational speed of the wire devices 30a and 30b and a value of a torque limiter, and prevents the wire devices 30a and 30b from operating at too high a speed or torque.

When the power source is turned on, the actuating apparatus 10 receives type information of the endoscope in connection with the processing apparatus 8 in a form of an output signal. The actuating apparatus 10 includes an inner storage medium. The actuating apparatus 10 recognizes the type information of the endoscope for use and type information of the propulsion assembly 2 by referring to table data stored in the storage medium. The table data is data of types of the endoscope and usable types of the propulsion assembly 2 in association with the endoscope types. For example, a shiftable range of the sealing device 21 is determined according to the type information of the propulsion assembly 2. An outer diameter of the tip device 3 is determined according to the type information of the endoscope. It is possible promptly to check whether the propulsion assembly 2 can be properly used in connection with the tip device 3 of the endoscope. If it is judged that a combination of the propulsion assembly 2 with the tip device 3 is improper, an alarm signal is generated, for example, alarm sound or visible alarm sign of light with an alarm lamp. Also, operation of the propulsion assembly 2 may be inhibited. Those functions can prevent occurrence of accidents.

When a foot switch 11 in connection with the actuating apparatus 10 is depressed, the motors in the actuating apparatus 10 rotate to apply torque to the wire devices 30a and 30b. The coupling gears 32a and 32b are caused to rotate, so that the spur gear teeth 24b meshed with the first coupling gear 32a are rotated with the drive sleeve 24. The second coupling gear 32b rotates in a direction opposite to that of the first coupling gear 32a. Rotation of the second coupling gear 32b is directly transmitted to the first coupling gear 32a. Thus, the motors in the actuating apparatus 10 can be utilized to rotate the drive sleeve 24.

When the worm gear teeth 24a of the drive sleeve 24 rotate, the worm wheels 27 rotate in the same direction about respectively the gear shaft 27a. The endless track device 15 is tensioned between the teeth of the worm wheels 27 and the idler rollers 42 of the roller mechanisms 40. The idler rollers 42 are caused to rotate by the worm wheels 27 to move the endless track device 15 endlessly in the axial direction of the drive sleeve 24. In FIG. 5, the worm wheels 27 rotate in the clockwise direction. The idler rollers 42 rotate in the counterclockwise direction. A return run 80 of the endless track device 15 inside the outer sleeve unit 17 moves from the proximal side to the distal side. A working run 82 of the endless track device 15 outside the outer sleeve unit 17 moves from the distal side to the proximal side. Thus, the endless track device 15 endlessly turns around in the direction Y.

The working run 82 of the endless track device 15 contacts a wall of the large intestine in entry of the endoscope with the propulsion assembly 2 in the gastrointestinal tract. While the endless track device 15 endlessly moves, propulsion force for advancing the tip device 3 is obtained, in other words, force for pressing the wall of the large intestine in the proximal direction is obtained. During the distal movement of the endoscope, foreign material stuck on the working run 82 of the endless track device 15 may move toward the position of the return run 80 after passing the proximal end of the outer sleeve unit 17. However, the flange edge of the proximal cover flange 19b is positioned very close to the endless track device 15 and prevents the foreign material from internal jamming. Also, the proximal cover flange 19b prevents tissue of a body part from internal jamming together with the endless track device 15. Note that during the proximal movement of the endoscope, the flange edge of the distal cover flange 19a operates in the same manner for protection.

If the operator wishes to remove the propulsion assembly 2 from the tip device 3, the clamping sleeve 20 is rotated in the counterclockwise direction by use of the screw driving device. The clamping sleeve 20 shifts in an outward direction by rotating, and releases the sealing device 21 from being pressed. The sealing device 21 is enlarged by its resiliency to separate its inner surface from an outer surface of the tip device 3. The propulsion assembly 2 can be removed from the endoscope easily.

In the above embodiment, the screw driving device 63 has the thumb wheel portion 63a. However, a grip portion of the screw driving device 63 may have another shape, for example, a polygonal shape, handle shape, T-shape, wing shape or the like . The screw driving device 63 can be a torque generator for rotating electrically.

The driving head 63b of the screw driving device 63, although in a sleeve shape having the central hole in the above embodiment, may have a shape of a shaft without hollowness.

Although the teeth for mesh with one another are used in the mounting structure 62 of the clamping sleeve 20 and the driving head 63b of the screw driving device 63, structures of other well-known shapes for those can be used. For example, the mounting structure 62 may have internal gear teeth on an inner surface of the clamping sleeve 20. The driving head 63b may have spur gear teeth on its outer surface. Also, the mounting structure 62 may have spur gear teeth on an outer surface of the clamping sleeve 20. The driving head 63b may have internal gear teeth on an inner surface of its central hole. Also, the mounting structure 62 may be a bolt head of a polygonal shape on the outer side of the clamping sleeve 20. The driving head 63b may have a socket or recess of a wrench of a polygonal shape on an inner surface of its central hole.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A propulsion assembly having an endless track device for endlessly moving in contact with a wall of a body cavity, for propulsion of a tip device of an endoscope in said body cavity, comprising:
a support sleeve for mounting on said tip device, said endless track device extending around said support sleeve;
a clamping sleeve contained in said support sleeve;
a male thread formed on an outer surface of said clamping sleeve;
a female thread, formed on an inner surface of said support sleeve, engaged helically with said male thread, for shifting said clamping sleeve between operative and non-operative states upon rotation of said clamping sleeve;
a receiving surface, formed inside said support sleeve, and disposed on a proximal side from said clamping sleeve in said operative state;
a sealing device, contained in said support sleeve, having a C-shape defined arcuately according to an annular shape in a resiliently deformable manner, disposed around said tip device and between said clamping sleeve and said receiving surface, wherein said sealing device is pushed by said clamping sleeve when said clamping sleeve is in said operative state, for squeezing said tip device by radially compressing around said tip device, and released from push of said clamping sleeve when said clamping sleeve is in said non-operative state, for releasing said tip device by radially returning to expand around said tip device.

2. A propulsion assembly as defined in claim 1, further comprising:
a first tapered surface, formed on an outer side of said sealing sleeve, and tapered relative to a central axis of said tip device;
a second tapered surface, formed on an inner side of said clamping sleeve, tapered relative to said central axis, for pushing said first tapered surface when in said operative state, to compress said sealing device, and for coming away from said first tapered surface when in said non-operative state, to allow said sealing device to expand.

3. A propulsion assembly as defined in claim 2, further comprising a mounting structure, disposed at an end of said clamping sleeve in a distal direction, for engagement with a jig device for rotating said clamping sleeve, to be supplied with torque.

4. A propulsion assembly as defined in claim 1, further comprising a first tapered surface, formed on an outer side of said sealing sleeve, and tapered relative to said central axis, wherein said first tapered surface, when said clamping sleeve is in said operative state, is pushed by said clamping sleeve for compressing said sealing device, and when said clamping sleeve is in said non-operative state, is released from said clamping sleeve coming away, and allows said sealing device to expand.

5. A propulsion assembly as defined in claim 1, further comprising a tapered surface, formed on an inner side of said clamping sleeve, tapered relative to said central axis, for pushing said sealing device when in said operative state, to compress said sealing device, and for coming away from said sealing device when in said non-operative state, to allow said sealing device to expand.

6. A fastening method for a propulsion assembly including an endless track device for endlessly moving in contact with a wall of a body cavity, for propulsion of a tip device of an endoscope in said body cavity, and a support sleeve for mounting on said tip device, said endless track device extending around said support sleeve, said fastening method comprising steps of:
entering said tip device in said support sleeve by entry of said tip device in a sealing device contained in said support sleeve and having a C-shape in a manner deformable resiliently in a radial direction;
rotating said clamping sleeve in said support sleeve by use of helical engagement between a female thread formed on an inner surface of said support sleeve and a male thread formed on an outer surface of said clamping sleeve, to move said clamping sleeve in a proximal direction along a central axis of said tip device;
pushing said sealing device with said clamping sleeve moved in said proximal direction, to compress said sealing device in said radial direction around said tip device, to squeeze said tip device.

7. A fastening method as defined in claim 6, wherein said sealing device includes a first tapered surface at an end thereof in a distal direction, and said clamping sleeve includes a second tapered surface at an end thereof in said proximal direction, for pushing said first tapered surface.

8. A fastening method as defined in claim 6, wherein a jig device is used and accesses a distal portion of said propulsion assembly, to rotate said clamping sleeve relative to said support sleeve.

9. A fastening method as defined in claim 8, wherein said clamping sleeve has a mounting structure disposed at an end thereof in a distal direction, and said jig device includes a rotatable driving head, engaged with said mounting structure, for applying torque thereto.

10. A fastening method as defined in claim 9, wherein said driving head includes a plurality of first teeth, and said mounting structure includes a plurality of second teeth, meshed with said first teeth, for transmitting said torque.
